SEQUENCE LISTING (18 sequences)

SEQ ID NO: 1 - protein, 129 aa, Mus musculus
DIVMTQSHKFMSTSVGDRVSITCKASQDVSSAVAWYQQKPGQSPKLLIYSASYRYTGVPDRFTGSGSGTDFTFTISSVQAEDLAVYYCQQHYSSPRTFGGGTNLEIKRADAAPTVSIFPPSSEQLTSGG

SEQ ID NO: 2 - DNA, 389 bp, Mus musculus
gatattgttatgactcagtctcacaaattcatgtccacatcagtaggagacagagtcagcatcacctgcaaggccagtcaggatgtgagttctgctgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtagtcctcggacgttcggtggaggcaccaacctggaaatcaaacgggctgatgctgcaccaactgtatccatcttcccaccatccagtgagcagttaacatctggaggtgc

SEQ ID NO: 3 - protein, 134 aa, Mus musculus
EVQLVESGGDLVKPGGSLKLSCAASGFTFSRYGMSWVRQTPDKRLEWVATISSGGSYTYYPDSVKGRFTISRDNAKNTLYLQMSSLKSEDTAMYYCARLDGYNYRWYFDVWGTGTTVTVSSAKTTPPSVYPLAP

SEQ ID NO: 4 - DNA, 402 bp, Mus musculus
gaggttcagctcgttgagtctgggggagacttagtgaagcctggagggtccctgaaactctcctgtgcagcctctggattcactttcagtaggtatggcatgtcttgggttcgccagactccagacaagaggctggagtgggtcgcaaccattagtagtggtggtagttacacctactatccagacagtgtgaaggggcgattcaccatctccagagacaatgccaagaacaccctgtacctgcaaatgagcagtctgaagtctgaggacacagccatgtattactgtgcaagactagacggttataactacaggtggtacttcgatgtctggggcacagggaccacggtcaccgtctcctcagccaaaacaacacccccatcagtctatccactggcccct

SEQ ID NO: 5 - protein, 11 aa, Mus musculus
KASQDVSSAVA

SEQ ID NO: 6 - protein, 7 aa, Mus musculus
SASYRYT

SEQ ID NO: 7 - protein, 9 aa, Mus musculus
QQHYSSPRT

SEQ ID NO: 8 - protein, 5 aa, Mus musculus
RYGMS

SEQ ID NO: 9 - protein, 17 aa, Mus musculus
TISSGGSYTYYPDSVKG

SEQ ID NO: 10 - protein, 12 aa, Mus musculus
LDGYNYRWYFDV

SEQ ID NO: 11 - DNA, 17 bp, Mus musculus
gatattgttatgactca

SEQ ID NO: 12 - DNA, 17 bp, Mus musculus
gaggttcagctcgttga

SEQ ID NO: 13 - DNA, 33 bp, Mus musculus
aaggccagtcaggatgtgagttctgctgtagcc

SEQ ID NO: 14 - DNA, 21 bp, Mus musculus
tcggcatcctaccggtacact

SEQ ID NO: 15 - DNA, 27 bp, Mus musculus
cagcaacattatagtagtcctcggacg

SEQ ID NO: 16 - DNA, 15 bp, Mus musculus
aggtatggcatgtct

SEQ ID NO: 17 - DNA, 51 bp, Mus musculus
accattagtagtggtggtagttacacctactatccagacagtgtgaagggg

SEQ ID NO: 18 - DNA, 36 bp, Mus musculus
ctagacggttataactacaggtggtacttcgatgtc

(19) United States
(12) Patent Application Publication
(10) Pub. No.: US 2004/0213788 A1
(43) Pub. Date: Oct. 28, 2004

(54) Anti-rank ligand monoclonal antibodies useful in treatment of rank ligand mediated disorders

(76) Inventors: Raymond W. Sweet, Bala Cynwyd, PA (US); Mark A. Tornetta, Malvern, PA (US); Trevor A. Wattam, Harlow Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/477,377

(22) PCT Filed: May 3, 2002
(86) PCT No.: PCT/US02/14246

(30) Foreign Application Priority Data
May 18, 2001 (US) 60297031

(51) Int. Cl. A61K 39/395; C07H 21/04; C07K 16/40; C12N 5/06

(52) U.S. Cl. 424/146.1; 530/388.26; 536/23.53; 435/69.1; 435/320.1; 435/338

(57) ABSTRACT

Chimeric, humanized and other RANK-L mAbs, derived from high affinity neutralizing mAbs, pharmaceutical compositions containing same, methods of treatment and diagnostics are provided.

### CROSS-REFERENCE TO RELATED APPLICATION

This application claims benefit to the earlier provisional U.S. Application No. 60/292,031, filed on May 18, 2001, the contents of which are incorporated herein by reference in their entirety.

### FIELD OF THE INVENTION

The present invention relates generally to the field of antibodies useful in the treatment and diagnosis of conditions mediated by RANK Ligand, and more specifically, to mAbs, Fabs, altered, chimeric, and humanized antibodies.

### BACKGROUND OF THE INVENTION

Human RANK Ligand (RANK-L) is a member of the tumor necrosis factor superfamily of proteins known to be key regulators of the immune system, bone development and homeostasis (Anderson, et al., Nature 390: 175-179, 1997). This ligand is also designated Tumor Necrosis Factor Related Activation-Induced Cytokine (TRANCE) (Wong,et al., J. Exp. Med. 186: 2075, 1997), Osteoprotegerin Ligand (OPGL) (Lacey, et al., Cell 93:165, 1998), and Osteoclast Differentiation Factor (ODF) (Yasuda, et al., Proc. Natl. Acad. Sci. 95: 3597, 1998). Members of the tumor necrosis family mediate diverse and sometimes opposite biological responses such as proliferation, apoptosis, cell survival, and differentiation. Other members of the TNF family of ligands described to date include 4-1BBL, APRIL, CD40L, CD30L, CD27L, FasL, LIGHT, LT-a, LT-b, OX40L, TNFa, Trail, RANK-L, and TWEAK (reviewed in Wong et al., J. Leukocyte Biol.: 65 715, 1999 and in Kwon et al., Curr Opin Immunol 11: 340, 1999). Among these other ligands, RANKL shares greatest homology to CD40L (about 28% identity in the extracellular region).

Like other members of the TNF ligand family, RANK-L is expressed as a type II membrane protein with a short cytoplasmic tail and an extracellular TNF core domain that comprises the binding site for the RANK-L receptor, RANK. The receptor binding domain can be proteolytically cleaved to release soluble RANKL capable of stimulating receptor function at a distance. This cleavage is blocked by inhibitors of metalloproteases, and purified TNF alpha converting enzyme (TACE) can induce cleavage, suggesting that this processing is mediated by TACE or a similar enzyme (Lum et al., J. Biol. Chem. 274: 13613, 1999). RANK-L is expressed on activated T-cells, activated osteoblasts, and bone marrow stromal cells providing a link between immune system biology and bone biology. Biochemical evidence shows that RANK-L is glycosylated. The cytoplasmic tail has motifs that may act as docking sites for SH3 domain containing proteins and accordingly may mediate reverse signaling upon binding to its receptor.

RANK-L receptors

Two receptors have been identified for RANK-L, RANK and OPG. RANK is a TNF receptor family member most closely related to CD40 (Anderson et al., Nature 390:175, 1997). RANK is a type I membrane receptor of 616 amino acids with a 184 amino acid extracellular domain, a transmembrane domain, and a large cytoplasmic domain of 383 amino acids. Although broadly expressed as mRNA, the expression of RANK protein on the cell surface appears to be limited to splenic, lymph node and bone marrow-derived dendritic cells and osteoclast progenitor cells (Wong et al., J. Exp. Med., 186:2075, 1997; Anderson et al., Nature 390:175, 1997; Lacey et al., Cell 93:165, 1998). Like many of the TNF receptor family members, the cytoplasmic domain of RANK is thought to mediate signal transduction through interaction with adaptor molecules known as TNF-receptor associated factors (TRAFs). TRAFs in turn activate several different pathways such as NF-kB and mitogen-induced protein kinases (MAPK) such as c-Jun amino-terminal protein kinases (JNK) and the extracellular signal-regulated kinases (ERK). These different signal transduction pathways variously mediate cell survival signals, apoptosis, differentiation, cytokine secretion, and/or cell activation. Accordingly, interaction of RANK-L and RANK may play a critical role in the regulation of immune function and bone homeostasis. Biochemical and genetic gene knockout studies indicate that the TRAF-6, and also TRAF-2 and TRAF-5, are the primary members of this family that associate with the cytoplasmic region of RANK.

The second identified RANK-L receptor is osteoprotegerin (OPG), which lacks a transmembrane region and appears to function as a soluble decoy receptor that acts to block signaling between RANK-L and its cognate cell surface receptor RANK. OPG is known to be a potent inhibitor of bone resorption and can inhibit RANK-L mediated osteoclastogenesis in vitro and in vivo (Lacey et al., Cell 93:165, 1998; Yasuda et al., PNAS 95:3597, 1998; Tomoyasu et al., Biochem. Biophys. Res. Commun. 245:382, 1998; Tsuda and Higashio, Nippon Rinsho 56:1435, 1998). OPG also binds to the TNF ligand TRAIL (Emery et al., J. Biol. Chem. 273:14363, 1998). Role of RANK-L in dendritic cell biology.

Mature bone marrow dendritic cells and splenic dendritic cells express high levels of RANK on their surfaces suggesting a central role for RANK-L in regulation of dendritic cell biology (Wong et al., J. Exp. Med., 186:2075, 1997). One primary effect of RANK-L is to increase the survival of mature dendritic cells, perhaps through upregulation of Bcl-xL, a well described apoptotic suppressor (Wong et al., J. Exp. Med., 186:2075, 1997). Increased DC survival can in turn lead to enhanced T cell proliferative responses by prolonging the stimulatory presentation of antigen/MHC complexes and costimulatory molecules such as B7-1 and B7-2 (Wong et al., J. Exp. Med., 186:2075, 1997). Stimulation of dendritic cells by RANK-L is also known to induce transcription of several cytokine genes such as IL-12, IL-15, IL-1, and IL-6 (Wong et al., J. Leukocyte Biol. 65:715, 1999). These cytokines regulate the intensity and type of immune response. In a CD40L knockout background, residual viral resistance is mediated by the RANKL-RANK pathway (Bachman et al., J. Exp. Med. 189: 1017-1020). Also, RANKL and RANK knockout mice are deficient in lymph-node organogenesis and show some defects in early B and T cell development (Kong et al., Nature 397: 315, 1999; Dougall et al., Genes and Development 13:2412, 1999; Li et al., Proc. Natl Acad. Sci. 97:1566, 2000). Thus, RANK-L appears to play a role in development of the immune system and in modulation of the quality and intensity of the immune response.

Role of RANK-L in Bone Biology

RANK-L is the critical differentiation factor for the development and activation of osteoclasts and as such plays a major role in maintaining bone homeostasis and calcium metabolism. RANK-L can stimulate the differentiation of bone resorbing osteoclasts from myeloid precursors (Lacey et al., Cell 93:165, 1998; Yasuda et al., PNAS 95:3597, 1998). Thus, RANK-L and RANK knock-out mice were characterized by severe osteopetrosis due to a complete lack of osteoclast differentiation (Kong et al., Nature 397: 315, 1999; Dougall et al., Genes and Development 13:2412, 1999; Li et al., Proc. Natl. Acad. Sci. 97:1566, 2000). Moreover, systemic overexpression of the RANK-L decoy receptor OPG in transgenic mice similarly was found to cause osteopetrosis (Simonet et al., Cell 89:309, 1997) as does systemic administration of soluble RANK ectodomain protein (Fuller et al., J. Exp. Med. 188:997, 1998). RANK-L also stimulates osteoclasts resulting in increased motility, spreading, and survival of mature osteoclasts. This stimulation in turn results in more efficient bone resorption by the activated osteoclasts. Thus, it appears that bone homeostasis depends at least in part on the balance of expression of RANK-L and OPG. Accordingly, diseases of bone may be treated by increasing or decreasing the action of RANK-L. For example, activated T cells upregulate expression of RANKL (Josien et al., J. Immunol 162: 2562-2568, 1999; Kong et al., Nature 402: 304-309, 1999) and polyclonal activated T cells from ctla4 knockout mice induce bone loss upon adoptive transfer into rag knockout mice that is inhibited by administration of OPG (Kong et al., Nature 402: 304-309, 1999). Also, in adjuvant induced arthritis in the rat, administration of OPG protein inhibits bone and cartilage loss without effect on the inflammatory reaction (Kong et al., Nature 402: 304-309, 1999).

In summary, ligation of RANK with RANK-L results in osteoclast differentiation and osteoclast activation in the bone marrow or dendritic cell survival and cytokine production in the lymphoid organs leading to increased bone resorption and enhanced immune responses, respectively. Accordingly, RANK-L is a desirable target for the development of a novel therapeutic for immune system disorders and diseases of bone homeostasis.

Neutralizing RANK-L antibodies may be useful in relieving pathological bone loss and related symptoms in man. Neutralizing RANK-L antibodies may also be useful in relieving inflammatory and autoimmune diseases and related symptoms in man. Hence, there is also a need in the art for neutralizing monoclonal antibodies to human RANK-L, which would reduce RANK-L mediated osteoclast differentiation and activation and thus diseases of the bone and related symptoms. There is also a need in the art for a high affinity RANK-L antagonist, such as a neutralizing monoclonal antibody to human RANK-L, which would reduce RANK-L mediated potentiation of immune responses and thus diseases of the immune system and related symptoms. Antagonist RANKL monoclonal antibodies are expected to be more selective in their action than OPG proteins which have the potential to interact with other TNF related ligands such as TRAIL.

### SUMMARY OF THE INVENTION

In a first aspect, the present invention provides neutralizing monoclonal antibodies specific for human RANK-L and having a binding affinity characterized by a dissociation constant equal to or less than about 10⁻¹⁰ M as described in the detailed description. Exemplary of such monoclonal antibodies is the mouse monoclonal antibody 14F3. Another aspect of the invention is the hybridoma 2413 14F3(2)B11. In a related aspect, the present invention provides neutralizing Fab fragments or F(ab′)₂ fragments thereof specific for human RANK-L produced by deleting the Fc region of the rodent neutralizing monoclonal antibodies of the present invention.

In still another related aspect, the present invention provides an altered antibody specific for human RANK-L which comprises complementarity determining regions (CDRs) derived from a non-human neutralizing monoclonal antibody (mAb) characterized by a dissociation constant equal to or less than about 10⁻¹⁰ M for human RANK-L and nucleic acid molecules encoding the same. When the altered antibody is a humanized antibody, the sequences that encode complementarity determining regions (CDRs) from a non-human immunoglobulin are inserted into a first immunoglobulin partner in which at least one, and preferably all complementarity determining regions (CDRs) of the first immunoglobulin partner are replaced by CDRs from the non-human monoclonal antibody. Preferably, the first immunoglobulin partner is operatively linked to a second immunoglobulin partner as well, which comprises all or a part of an immunoglobulin constant chain.

In a related aspect, the present invention provides CDRs derived from non-human neutralizing monoclonal antibodies (mAbs) characterized by a dissociation constant equal to or less than about 10⁻¹⁰ M for human RANK-L, and nucleic acid molecules encoding such CDRs.

In still another aspect, there is provided a chimeric antibody containing human heavy and light chain constant regions and heavy and light chain variable regions derived from non-human neutralizing monoclonal antibodies characterized by a dissociation constant equal to or less than about 10⁻¹⁰M for human RANK-L.

In yet another aspect, the present invention provides a pharmaceutical composition which contains one (or more) of the above described antibodies and a pharmaceutically acceptable carrier.

In a further aspect, the present invention provides a method for treating conditions in humans associated with excess RANK-L, for diseases of the immune system or bone, in particular, osteopenic diseases, including rheumatoid arthritis (RA), osteoporosis (OP), metastatic and primary bone cancer, wear debris induced osteolysis or osteoarthritis (OA), and immune diseases, including psoriasis, insulin dependent, diabetes (IDDM), inflammatory bowel disease (IBD), or multiple sclerosis (MS), by administering to said human an effective amount of the pharmaceutical composition of the invention.

In yet another aspect, the present invention provides methods for, and components useful in, the recombinant production of altered antibodies (e.g., engineered antibodies, CDRs, Fab or F(ab)₂ fragments, or analogs thereof) which are derived from non-human neutralizing monoclonal antibodies (mAbs) characterized by a dissociation constant equal to or less than 10⁻¹⁰ M for human RANK-L. These components include isolated nucleic acid sequences encoding same, recombinant plasmids containing the nucleic acid sequences under the control of selected regulatory sequences which are capable of directing the expression thereof in host cells (preferably mammalian) transfected with the recombinant plasmids. The production method involves culturing a transfected host cell line of the present invention under conditions such that an antibody, preferably a humanized antibody, is expressed in said cells and isolating the expressed product therefrom.

In yet another aspect of the invention is a method to diagnose conditions associated with excess Th1 T cell activity or osteoclast development and activation, in particular osteopenic diseases, including rheumatoid arthritis (RA), osteoporosis (OP), metastatic and primary bone cancer, wear debris induced osteolysis or osteoarthritis (OA), and immune diseases, including psoriasis, insulin dependent, diabetes (IDDM), inflammatory bowel disease (IBD), or multiple sclerosis (MS), in a human which comprises obtaining a sample of biological fluid from a patient and allowing the antibodies and altered antibodies of the instant invention to come in contact with such sample under conditions such that an RANK-L/antibody (monoclonal or altered) complex is formed and detecting the presence or absence of said RANK-L/antibody complex.

Other aspects and advantages of the present invention are described further in the detailed description and the preferred embodiments thereof.

### DETAILED DESCRIPTION OF THE INVENTION

Table I shows a cDNA of the light chain variable region and the deduced amino acid sequences for the mouse antibody 14F3(SEQ ID NOs: 1 and 2, respectively) The boxed areas (within the box of Table I) indicate three CDR's (SEQ ID NO: 5, 6 and 7) and respective polynucleotides encoding the CDR's (SEQ ID NO: 13, 14, and 15). The bolded area indicates the degenerate primer sequence (SEQ ID NO: 11).

TABLE I

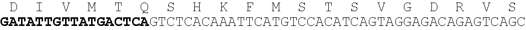

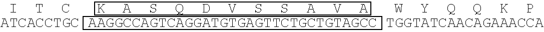

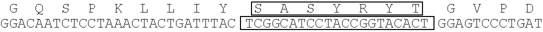

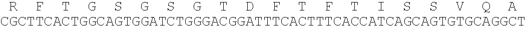

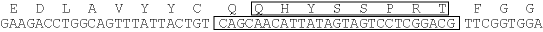

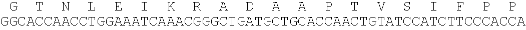

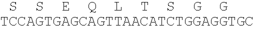

Table II shows cDNA of the heavy chain variable region and the deduced amino acid sequences for the mouse antibody 14F3 (SEQ ID NOs: 3 and 4, respectively) The boxed areas (within the box of Table II) indicate three CDR's (SEQ ID NOS: 8, 9, and 10), and respective polynucleotides encoding the CDR's (SEQ ID NOs: 16, 17, and 18). The bolded area indicates the degenerate primer sequence (SEQ ID NO: 12).

TABLE II

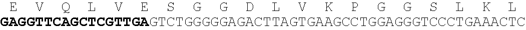

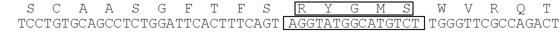

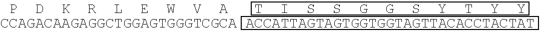

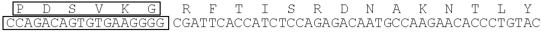

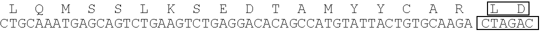

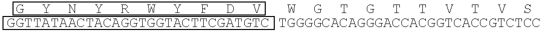

The present invention provides a variety of antibodies, altered antibodies and fragments thereof, which are characterized by human RANK-L binding specificity, neutralizing activity, and high affinity for human RANK-L as exemplified in mouse monoclonal antibody 14F3, for which variable light and heavy chain regions are provided in Tables I and II. The monoclonal antibody 14F3 was prepared by conventional hybridoma techniques to generate a novel neutralizing antibody. The antibodies of the present invention are useful in therapeutic and pharmaceutical compositions for treating RANK-L-mediated disorders, e.g. osteopenic diseases, including rheumatoid arthritis (RA), osteoporosis (OP), metastatic and primary bone cancer, wear debris induced osteolysis or osteoarthritis (OA), and immune diseases, including psoriasis, insulin dependent, diabetes (EDDM), inflammatory bowel disease (IBD), or multiple sclerosis (MS). This product is also useful in the diagnosis of RANK-L-mediated conditions by measurement (e.g., enzyme linked immunosorbent assay (ELISA)) of endogenous RANK-L levels in humans or RANK-L released ex vivo from activated cells.

I. Definitions.

“Antibodies” include, but are not limited to, monoclonal, altered, humanized, engineered, and chimeric antibodies.

“Monoclonal antibodies”, as opposed to polyclonal antibodies, refer to immunoglobulins which can be prepared by conventional hybridoma techniques, phage display combinatorial libraries, immunoglobulin chain shuffling and humanization techniques.

“Altered antibody” refers to a protein encoded by an altered immunoglobulin coding region, which may be obtained by expression in a selected host cell. Such altered antibodies are engineered antibodies (e.g., chimeric or humanized antibodies) or antibody fragments lacking all or part of an immunoglobulin constant region, e.g., Fv, Fab, or F(ab)₂ and the like.

“Altered immunoglobulin coding region” refers to a nucleic acid sequence encoding altered antibody of the invention. When the altered antibody is a CDR-grafted or humanized antibody, a first immunoglobulin partner comprising human variable framework sequences are replaced by the sequences that encode the complementarity determining regions (CDRs) from a non-human immunoglobulin. Optionally, the first immunoglobulin partner is operatively linked to a second immunoglobulin partner.

“First immunoglobulin partner” refers to a nucleic acid sequence encoding a human framework or human immunoglobulin variable region in which the native (or naturally-occurring) CDR-encoding regions are replaced by the CDR-encoding regions of a donor antibody. The human variable region can be an immunoglobulin heavy chain, a light chain (or both chains), an analog or functional fragments thereof. Such CDR regions, located within the variable region of antibodies (immunoglobulins) can be determined by known methods in the art. For example Kabat et al. (Sequences of Proteins of Immunological Interest, 4th Ed., U.S. Department of Health and Human Services, National Institutes of Health (1987)) disclose rules for locating CDRs. In addition, computer programs are known which are useful for identifying CDR regions/structures.

“Neutralizing” refers to an antibody that inhibits RANK-L activity by preventing the binding of human RANK-L to its specific receptor or by inhibiting the signaling of RANK-L through its receptor, should binding occur. A mAb is neutralizing if it is 90% effective, preferably 95% effective and most preferably 100% effective in inhibiting RANK-L activity as measured in the RANK-L neutralization assay.

The term “high affinity” refers to an antibody having a binding affinity characterized by a K_{d} equal to or less than 10⁻¹⁰ M for human RANK-L as determined by optical biosensor analysis.

By “binding specificity for human RANK-L” is meant a higher affinity for human RANK-L than murine, or other RANK-L.

“Second immunoglobulin partner” refers to another nucleotide sequence encoding a protein or peptide to which the first immunoglobulin partner is fused in frame or by means of an optional conventional linker sequence (i.e., operatively linked). Preferably it is an immunoglobulin gene. The second immunoglobulin partner may include a nucleic acid sequence encoding the entire constant region for the same (i.e., homologous—the first and second altered antibodies are derived from the same source) or an additional (i.e., heterologous) antibody of interest. It may be an immunoglobulin heavy chain or light chain (or both chains as part of a single polypeptide). The second immunoglobulin partner is not limited to a particular immunoglobulin class or isotype. In addition, the second immunoglobulin partner may comprise part of an immunoglobulin constant region, such as found in a Fab, or F(ab)₂ (i.e., a discrete part of an appropriate human constant region or framework region). Such second immunoglobulin partner may also comprise a sequence encoding an integral membrane protein exposed on the outer surface of a host cell, e.g., as part of a phage display library, or a sequence encoding a protein for analytical or diagnostic detection, e.g., horseradish peroxidase, β-galactosidase, etc.

The terms Fv, Fc, Fd, Fab, or F(ab)₂ are used with their standard meanings (see, e.g., Harlow et al., Antibodies A Laboratory Manual, Cold Spring Harbor Laboratory, (1988)).

As used herein, an “engineered antibody” describes a type of altered antibody, i.e., a full-length synthetic antibody (e.g., a chimeric or humanized antibody as opposed to an antibody fragment) in which a portion of the light and/or heavy chain variable domains of a selected acceptor antibody are replaced by analogous parts from one or more donor antibodies which have specificity for the selected epitope. For example, such molecules may include antibodies characterized by a humanized heavy chain associated with an unmodified light chain (or chimeric light chain), or vice versa. Engineered antibodies may also be characterized by alteration of the nucleic acid sequences encoding the acceptor antibody light and/or heavy variable domain framework regions in order to retain donor antibody binding specificity. These antibodies can comprise replacement of one or more CDRs (preferably all) from the acceptor antibody with CDRs from a donor antibody described herein.

A “chimeric antibody” refers to a type of engineered antibody which contains naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl. Acad Sci USA, 86:10029-10032 (1989), Hodgson et al., Bio/Technology 9:421 (1991)).

The term “donor antibody” refers to an antibody (monoclonal, or recombinant) which contributes the nucleic acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody. One donor antibody suitable for use in this invention is a non-human neutralizing monoclonal antibody designated as 14F3. The antibody 14F3 is defined as a high affinity, human-RANK-L specific (i.e., does not recognize murine RANK-L), neutralizing antibody of isotype IgG2a/kappa. This antibody has the variable light chain DNA and amino acid sequences of SEQ ID NOs: 1 and 2, respectively; and the variable heavy chain DNA and amino acid sequences of SEQ ID NOs: 3 and 4, respectively, on a suitable murine IgG constant region.

The term “acceptor antibody” refers to an antibody (monoclonal, or recombinant) heterologous to the donor antibody, which contributes all (or any portion, but preferably all) of the nucleic acid sequences encoding its heavy and/or light chain framework regions and/or its heavy and/or light chain constant regions to the first immunoglobulin partner. Preferably a human antibody is the acceptor antibody.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate).

CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

By sharing the antigen binding specificity or neutralizing ability is meant, for example, that although mAb 14F3 may be characterized by a certain level of antigen affinity, a CDR encoded by a nucleic acid sequence of 14F3 in an appropriate structural environment may have a lower, or higher affinity. It is expected that CDRs of 14F3 in such environments will nevertheless recognize the same epitope(s) asl4F3. Exemplary light chain CDRs of 14F3 include

SEQ ID NO: 5;

SEQ ID NO: 6;

SEQ ID NO: 7;

and exemplary heavy chain CDRs of 14F3 include

SEQ ID NO: 8;

SEQ ID NO: 9;

and SEQ ID NO: 10.

A “functional fragment” is a partial heavy or light chain variable sequence (e.g., minor deletions at the amino or carboxy terminus of the immunoglobulin variable region) which retains the same antigen binding specificity and/or neutralizing ability as the antibody from which the fragment was derived.

An “analog” is an amino acid sequence modified by at least one amino acid, wherein said modification can be chemical or a substitution or a rearrangement of a few amino acids (e.g., preferably no more than 10), which modification permits the amino acid sequence to retain the biological characteristics, e.g., antigen specificity and high affinity, of the unmodified sequence. For example, (silent) mutations can be constructed, via substitutions, when certain endonuclease restriction sites are created within or surrounding CDR-encoding regions.

Analogs may also arise as allelic variations. An “allelic variation or modification” is an alteration in the nucleic acid sequence encoding the amino acid or peptide sequences of the invention. Such variations or modifications may be due to degeneracy in the genetic code or may be deliberately engineered to provide desired characteristics. These variations or modifications may or may not result in alterations in any encoded amino acid sequence.

The concept of fragments, analogs, and allelic variation can also be represented in terms of “identity.” For examples, the present invention relates to polynucleotides or polypeptides which comprise polynucleotides or polypeptides which are at least 90%, even more preferably 95%, identical to a member selected from the group consisting of SEQ ID NOs: 1, 2, 3, 4, 5, 6, 7, 8, 9, 10, 13, 14, 15, and 16, 17, and 18.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12 (1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Preferred parameters for polypeptide sequence comparison include the following:

1) Algorithm: Needleman and Wunsch, J. Mol. Biol. 48: 443-453 (1970) Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman and Wunsch, J. Mol. Biol. 48: 443-453 (1970)

Comparison matrix: matches=+10, mismatch=0

Gap Penalty: 50

Gap Length Penalty: 3

Available as: The “gap” program from Genetics Computer Group, Madison Wis.

These are the default parameters for nucleic acid comparisons.

By way of example, a polynucleotide sequence of the present invention may be identical to the reference sequence of SEQ ID NO: 1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO: 1 by the numerical percent of the respective percent identity (divided by 100) and subtracting that product from said total number of nucleotides in SEQ ID NO: 1, or:

nn≦xn−(xn·y),

wherein nn is the number of nucleotide alterations, xn is the total number of nucleotides in SEQ ID NO: 1, and y is, for instance, 0.70 for 70%, 0.80 for 80%, 0.85 for 85%, 0.90 for 90%, 0.95 for 95%, etc., and wherein any non-integer product of xn and y is rounded down to the nearest integer prior to subtracting it from xn. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the numerical percent of the respective percent identity (divided by 100) and then subtracting that product from said total number of amino acids in SEQ ID NO:2, or:

na≦xa−(xa·y),

wherein na is the number of amino acid alterations, xa is the total number of amino acids in SEQ ID NO:2, and y is, for instance 0.70 for 70%, 0.80 for 80%, 0.85 for 85% etc., and wherein any non-integer product of xa and y is rounded down to the nearest integer prior to subtracting it from xa.

The term “effector agents” refers to non-protein carrier molecules to which the altered antibodies, and/or natural or synthetic light or heavy chains of the donor antibody or other fragments of the donor antibody may be associated by conventional means. Such non-protein carriers can include conventional carriers used in the diagnostic field, e.g., polystyrene or other plastic beads, polysaccharides, e.g., as used in the BIAcore [Pharmacia] system, or other non-protein substances useful in the medical field and safe for administration to humans and animals. Other effector agents may include a macrocycle, for chelating a heavy metal atom, or radioisotopes. Such effector agents may also be useful to increase the half-life of the altered antibodies, e.g., polyethylene glycol.

II. High Affinity RANK-L Monoclonal Antibodies

For use in constructing the antibodies, altered antibodies and fragments of this invention, a non-human species (for example, bovine, ovine, monkey, chicken, rodent (e.g., murine and rat), etc.) may be employed to generate a desirable immunoglobulin upon presentment with native human RANK-L or a peptide epitope therefrom. Conventional hybridoma techniques are employed to provide a hybridoma cell line secreting a non-human mAb RANK-L. Such hybridomas are then screened for binding using RANK-L coated to 96-well plates, as described in the Examples section, or alternatively with biotinylated RANK-L bound to a streptavidin coated plate.

One exemplary, high affinity, neutralizing mAb of this instant invention is mAb 14F3 (whose heavy and light chain variable regions are provided in Tables I and II), a mouse antibody which can be used for the development of a chimeric or humanized antibody, described in more detail in examples below. The 14F3 mAb is characterized by an antigen binding specificity for human IL-1 RANK-L of about Kd 10⁻¹⁰ M. This mAB is characterized by being isotype IgG2a/kappa.

This invention is not limited to the use of the 14F3 or its hypervariable (i.e., CDR) sequences. Any other appropriate high affinity RANK-L antibodies characterized by a dissociation constant equal or less than about 10⁻¹⁰ M for human RANK-L and corresponding anti-RANK-L CDRs may be substituted therefor. Wherever in the following description the donor antibody is identified as 14F3, this designation is made for illustration and simplicity of description only.

III. Antibody Fragments

The present invention also includes the use of Fab fragments or F(ab′)₂ fragments derived from mAbs directed against human RANK-L. These fragments are useful as agents protective in vivo against RANK-L and Th1 T cell mediated conditions, or in vitro as part of an RANK-L diagnostic, in particular osteopenic diseases, including rheumatoid arthritis (RA), osteoporosis (OP), metastatic and primary bone cancer, wear debris induced osteolysis or osteoarthritis (OA), and immune diseases, including psoriasis, insulin dependent, diabetes (IDDM), inflammatory bowel disease (IBD), or multiple sclerosis (MS). A Fab fragment contains the entire light chain and amino terminal portion of the heavy chain; and an F(ab′)₂ fragment is the fragment formed by two Fab fragments bound by disulfide bonds. MAb 14F3 and other similar high affinity, RANK-L binding antibodies, provide sources of Fab fragments and F(ab′)₂ fragments which can be obtained by conventional means, e.g., cleavage of the mAb with the appropriate proteolytic enzymes, papain and/or pepsin, or by recombinant methods. These Fab and F(ab′)₂ fragments are useful themselves as therapeutic, prophylactic or diagnostic agents, and as donors of sequences including the variable regions and CDR sequences useful in the formation of recombinant or humanized antibodies as described herein.

The Fab and F(ab′)₂ fragments can be constructed via a combinatorial phage library (see, e.g., Winter et al., Ann. Rev. Immunol., 12:433-455 (1994)) or via immunoglobulin chain shuffling (see, e.g., Marks et al., Bio/Technology, 10:779-783 (1992), which are both hereby incorporated by reference in their entirety) wherein the Fd or V_{H} immunoglobulin from a selected antibody (e.g., 14F3) is allowed to associate with a repertoire of light chain immunoglobulins, v_{L} (or v_{K}), to form novel Fabs. Conversely, the light chain immunoglobulin from a selected antibody may be allowed to associate with a repertoire of heavy chain immunoglobulins, v_{H} (or Fd), to form novel Fabs.

IV. Anti-RANK-L Amino Acid and Nucleotide Sequences of Interest

The mAb 14F3 or other antibodies described above may contribute sequences, such as variable heavy and/or light chain peptide sequences, framework sequences, CDR sequences, functional fragments, and analogs thereof, and the nucleic acid sequences encoding them, useful in designing and obtaining various altered antibodies which are characterized by the antigen binding specificity of the donor antibody.

As one example, the present invention provides variable light chain and variable heavy chain sequences from the RANK-L mAb 14F3 and sequences derived therefrom.

The nucleic acid sequences of this invention, or fragments thereof, encoding the variable light chain and heavy chain peptide sequences are also useful for mutagenic introduction of specific changes within the nucleic acid sequences encoding the CDRs or framework regions, and for incorporation of the resulting modified or fusion nucleic acid sequence into a plasmid for expression.

Taking into account the degeneracy of the genetic code, various coding sequences may be constructed which encode the variable heavy and light chain amino acid sequences, and CDR sequences of the invention as well as functional fragments and analogs thereof which share the antigen specificity of the donor antibody. The isolated nucleic acid sequences of this invention, or fragments thereof, encoding the variable chain peptide sequences or CDRs can be used to produce altered antibodies, e.g., chimeric or humanized antibodies, or other engineered antibodies of this invention when operatively combined with a second immunoglobulin partner.

In one embodiment, the present invention relates to polynucleotides or polypeptides which comprise polynucleotides or polypeptides which are at least 90%, even more preferably 95%, identical a member selected from the group consisting of SEQ ID NOs: 1, 2, 3, 4, 5, 6, 7, 8, 9, 10, 13, 14, 15, 16, 17, and 18. In another embodiment, the present invention relates to polynucleotides or polypeptides selected from the group consisting of SEQ ID NOs: 1, 2, 3, 4, 5, 6, 7, 8, 9, 10, 13, 14, 15, 16, 17, and 18. Yet in another embodiment, the invention relates to polynucleotides comprising polynucleotides at least 90%, even more preferably at least 95%, identical to the polynucleotides which encode the amino acid sequences selected from the group consisting of SEQ ID NOs: 2, 4, 5, 6, 7, 8, 9 and 10.

The present invention also relates to an antibody which comprises the polypeptides having the amino acid sequences of SEQ ID NOs:5, 6, 7, 8, 9 and 10. Further, the present invention also relates to an antibody which comprises the polypeptides having the amino acid sequences of SEQ ID NOs: 2 and 4. Also included within the scope of this invention are polynucleotides which encode an antibody comprising the polypeptides having the amino acid sequences of SEQ ID NOs:5, 6, 7, 8, 9 and 10. Further included are polynucleotides which encode an antibody comprising the polypeptides having the amino acid sequences of SEQ ID NOs: 2 and 4.

Also included are expression systems comprising polynucleotides which encode an antibody comprising the polypeptides having the amino acid sequences of SEQ ID NOs:5, 6, 7, 8, 9 and 10 capable of producing such antibody when said expression vectors are present in a compatible host cell, and recombinant host cells comprising such expression vectors. Also included are a process for producing an antibody which comprises the polypeptides having the amino acid sequencers of SEQ ID NOs:5, 6, 7, 8, 9 and 10 comprising the step of culturing said host cells under conditions sufficient for the production of said antibody and recovering the antibody from the culture medium.

Also included are expression systems comprising polynucleotides which encode an antibody comprising the polypeptides having the amino acid sequences of SEQ ID NOs: 2 and 4 capable of producing such antibody when said expression vectors are present in a compatible host cell, and recombinant host cells comprising such expression vectors. Also included are a process for producing an antibody which comprises the polypeptides having the amino acid sequences of SEQ ID NOs:2 and 4 comprising the step of culturing said host cells under conditions sufficient for the production of said antibody and recovering the antibody from the culture medium.

The present invention also relates to an antibody which comprises a polypeptide having the amino acid sequences of SEQ ID NOs:5, 6, and 7. Further, the present invention also relates to an antibody which comprises a polypeptide having the amino acid sequence of SEQ ID NO: 2. Also included within the scope of this invention are polynucleotides which encode an antibody comprising a polypeptide having the amino acid sequences of SEQ ID NOs:5, 6, and 7. Further included are polynucleotides which encode an antibody comprising a polypeptide having the amino acid sequence of SEQ ID NO: 2.

Also included are expression vectors comprising polynucleotides which encode an antibody comprising a polypeptide having the amino acid sequences of SEQ ID NOs:5, 6, and 7 capable of producing such antibody when said expression vectors are present in a compatible host cell, and recombinant host cells comprising such expression vectors. Also included are a process for producing an antibody which comprises a polypeptide having the amino acid sequences of SEQ ID NOs:5, 6, and 7 comprising the step of culturing said host cells under conditions sufficient for the production of said antibody and recovering the antibody from the culture medium.

Also included are expression systems comprising polynucleotides which encode an antibody comprising a polypeptide having the amino acid sequence of SEQ ID NO:2 capable of producing such antibody said expression vectors are present in a compatible host cell, and recombinant host cells comprising such expression vectors. Also included are a process for producing an antibody which comprises a polypeptide having the amino acid sequence of SEQ ID NO:2 comprising the step of culturing said host cells under conditions sufficient for the production of said antibody and recovering the antibody from the culture medium.

The present invention also relates to an antibody which comprises a polypeptide having the amino acid sequences of SEQ ID NOs: 8, 9 and 10. Further, the present invention also relates to an antibody which comprises a polypeptide having the amino acid sequence of SEQ ID NO: 4. Also included within the scope of this invention are polynucleotides which encode an antibody comprising a polypeptide having the amino acid sequences of SEQ ID NOs: 8, 9 and 10. Further included are polynucleotides which encode an antibody comprising a polypeptide having the amino acid sequence of SEQ ID NO 4.

Also included are expression systems comprising polynucleotides which encode an antibody comprising a polypeptide having the amino acid sequences of SEQ ID NOs:8, 9, and 10 capable of producing such antibody when said expression vectors are present in a compatible host cell, and recombinant host cells comprising such expression vectors. Also included are a process for producing an antibody which comprises a polypeptide having the amino acid sequences of SEQ ID NOs:8, 9, and 10 comprising the step of culturing said host cells under conditions sufficient for the production of said antibody and recovering the antibody from the culture medium.

Also included are expression systems comprising polynucleotides which encode an antibody comprising a polypeptide having the amino acid sequence of SEQ ID NO:4 capable of producing such antibody when said expression vectors are present in a compatible host cell, and recombinant host cells comprising such expression vectors. Also included are a process for producing an antibody which comprises a polypeptide having the amino acid sequence of SEQ ID NO:4 comprising the step of culturing said host cells under conditions sufficient for the production of said antibody and recovering the antibody from the culture medium.

It should be noted that in addition to isolated nucleic acid sequences encoding portions of the altered antibody and antibodies described herein, other such nucleic acid sequences are encompassed by the present invention, such as those complementary to the native CDR-encoding sequences or complementary to the modified human framework regions surrounding the CDR-encoding regions. Useful DNA sequences include those sequences which hybridize to any of the polynucleotides disclosed herein under stringent hybridization conditions [see, T. Maniatis et al, Molecular Cloning (A Laboratory Manual), Cold Spring Harbor Laboratory (1982), pages 387 to 389] to the DNA sequences. An example of one such stringent hybridization condition is hybridization at 4×SSC at 65° C., followed by a washing in 0.1×SSC at 65° C. for an hour. Alternatively an exemplary stringent hybridization condition is in 50% formamide, 4×SSC at 42° C. Preferably, these hybridizing DNA sequences are at least about 18 nucleotides in length, i.e., about the size of a CDR.

V. Altered Immunoglobulin Molecules And Altered Antibodies

Altered immunoglobulin molecules can encode altered antibodies which include engineered antibodies such as chimeric antibodies and humanized antibodies. A desired altered immunoglobulin coding region contains CDR-encoding regions that encode peptides having the antigen specificity of an RANK-L antibody, preferably a high affinity antibody such as provided by the present invention, inserted into a first immunoglobulin partner (a human framework or human immunoglobulin variable region).

Preferably, the first immunoglobulin partner is operatively linked to a second immunoglobulin partner. The second immunoglobulin partner is defined above, and may include a sequence encoding a second antibody region of interest, for example an Fc region. Second immunoglobulin partners may also include sequences encoding another immunoglobulin to which the light or heavy chain constant region is fused in frame or by means of a linker sequence. Engineered antibodies directed against functional fragments or analogs of RANK-L may be designed to elicit enhanced binding with the same antibody.

The second immunoglobulin partner may also be associated with effector agents as defined above, including non-protein carrier molecules, to which the second immunoglobulin partner may be operatively linked by conventional means.

Fusion or linkage between the second immunoglobulin partners, e.g., antibody sequences, and the effector agent may be by any suitable means, e.g., by conventional covalent or ionic bonds, protein fusions, or hetero-bifunctional cross-linkers, e.g., carbodiimide, glutaraldehyde, and the like. Such techniques are known in the art and readily described in conventional chemistry and biochemistry texts.

Additionally, conventional linker sequences which simply provide for a desired amount of space between the second immunoglobulin partner and the effector agent may also be constructed into the altered immunoglobulin coding region. The design of such linkers is well known to those of skill in the art.

In addition, signal sequences for the molecules of the invention may be modified to enhance expression.

An exemplary altered antibody contains a variable heavy and/or light chain peptide or protein sequence having the antigen specificity of mAb14F3, e.g., the V_{H} and V_{L} chains. Still another desirable altered antibody of this invention is characterized by the amino acid sequence containing at least one, and preferably all of the CDRs of the variable region of the heavy and/or light chains of the mouse antibody molecule 14F3 with the remaining sequences being derived from a human source, or a functional fragment or analog thereof.

In still a further embodiment, the engineered antibody of the invention may have attached to it an additional agent. For example, the procedure of recombinant DNA technology may be used to produce an engineered antibody of the invention in which the Fc fragment or CH2 CH3 domain of a complete antibody molecule has been replaced by an enzyme or other detectable molecule (i.e., a polypeptide effector or reporter molecule).

The second immunoglobulin partner may also be operatively linked to a non-immunoglobulin peptide, protein or fragment thereof heterologous to the CDR-containing sequence, for example, having the antigen specificity of mouse 14F3. The resulting protein may exhibit both anti-RANK-L antigen specificity and characteristics of the non-immunoglobulin upon expression. That fusion partner characteristic may be, e.g., a functional characteristic such as another binding or receptor domain, or a therapeutic characteristic if the fusion partner is itself a therapeutic protein, or additional antigenic characteristics.

Another desirable protein of this invention may comprise a complete antibody molecule, having full length heavy and light chains, or any discrete fragment thereof, such as the Fab or F(ab′)₂ fragments, a heavy chain dimer, or any minimal recombinant fragments thereof such as an Fᵥ or a single-chain antibody (SCA) or any other molecule with the same specificity as the selected donor mAb, e.g., mAb14F3. Such protein may be used in the form of an altered antibody, or may be used in its unfused form.

Whenever the second immunoglobulin partner is derived from an antibody different from the donor antibody, e.g., any isotype or class of immunoglobulin framework or constant regions, an engineered antibody results. Engineered antibodies can comprise immunoglobulin (Ig) constant regions and variable framework regions from one source, e.g., the acceptor antibody, and one or more (preferably all) CDRs from the donor antibody, e.g., the anti-RANK-L antibody described herein. In addition, alterations, e.g., deletions, substitutions, or additions, of the acceptor mAb light and/or heavy variable domain framework region at the nucleic acid or amino acid levels, or the donor CDR regions may be made in order to retain donor antibody antigen binding specificity.

Such engineered antibodies are designed to employ one (or both) of the variable heavy and/or light chains of the RANK-L mAb (optionally modified as described) or one or more of the below-identified heavy or light chain CDRs. The engineered antibodies would be expected to be are neutralizing, i.e., they desirably block binding to the receptor of the RANK-L protein and they also block or prevent proliferation of RANK-L dependent cells.

Such engineered antibodies may include a humanized antibody containing the framework regions of a selected human immunoglobulin or subtype, or a chimeric antibody containing the human heavy and light chain constant regions fused to the RANK-L antibody functional fragments. A suitable human (or other animal) acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody.

Desirably the heterologous framework and constant regions are selected from human immunoglobulin classes and isotypes, such as IgG (subtypes 1 through 4), IgM, IgA, and IgE. However, the acceptor antibody need not comprise only human immunoglobulin protein sequences. For instance a gene may be constructed in which a DNA sequence encoding part of a human immunoglobulin chain is fused to a DNA sequence encoding a non-immunoglobulin amino acid sequence such as a polypeptide effector or reporter molecule.

One example of a particularly desirable humanized antibody would contain CDRs 14F3 inserted onto the framework regions of a selected human antibody sequence. For neutralizing humanized antibodies, one, two or preferably three CDRs from the RANK-L antibody heavy chain and/or light chain variable regions are inserted into the framework regions of the selected human antibody sequence, replacing the native CDRs of the latter antibody.

Preferably, in a humanized antibody, the variable domains in both human heavy and light chains have been engineered by one or more CDR replacements. It is possible to use all six CDRs, or various combinations of less than the six CDRs. Preferably all six CDRs are replaced. It is possible to replace the CDRs only in the human heavy chain, using as light chain the unmodified light chain from the human acceptor antibody. Still alternatively, a compatible light chain may be selected from another human antibody by recourse to the conventional antibody databases. The remainder of the engineered antibody may be derived from any suitable acceptor human immunoglobulin.

The engineered humanized antibody thus preferably has the structure of a natural human antibody or a fragment thereof, and possesses the combination of properties required for effective therapeutic use, e.g., treatment of RANK-L mediated inflammatory diseases in man, or for diagnostic uses.

It will be understood by those skilled in the art that an engineered antibody may be further modified by changes in variable domain amino acids without necessarily affecting the specificity and high affinity of the donor antibody (i.e., an analog). It is anticipated that heavy and light chain amino acids may be substituted by other amino acids either in the variable domain frameworks or CDRs or both.

In addition, the constant region may be altered to enhance or decrease selective properties of the molecules of the instant invention. For example, dimerization, binding to Fc receptors, or the ability to bind and activate complement (see, e.g., Angal et al., Mol. Immunol, 30:105-108 (1993), Xu et al., J. Biol. Chem, 269:3469-3474 (1994), Winter et al., EP 307,434-B).

An altered antibody which is a chimeric antibody differs from the humanized antibodies described above by providing the entire non-human donor antibody heavy chain and light chain variable regions, including framework regions, in association with human immunoglobulin constant regions for both chains. It is anticipated that chimeric antibodies which retain additional non-human sequence relative to humanized antibodies of this invention may elicit a significant immune response in humans.

Thus, in one embodiment, the present altered antibody comprises one or more polynucleotides or polypeptides which are at least 90%, even more preferably at least 95%, identical to a member selected from the group consisting of SEQ ID NOs: 1, 2, 3, 4, 5, 6, 7, 8, 9, 10, 13, 14, 15, 16, 17, and 18. In another embodiment, the present altered antibody comprises one or more polynucleotides which are at least 90%, even more preferably at least 95%, identical to polynucleotides which encode the amino sequences selected from the group consisting of SEQ ID NOs: 2, 4, 5, 6, 7, 8, 9 and 10.

Such antibodies could be useful in the prevention and treatment of RANK-L mediated disorders, as discussed below.

VI. Production Of Altered Antibodies And Engineered Antibodies

Preferably, the variable light and/or heavy chain sequences and the CDRs of mAb 14F3 or other suitable donor mAbs, and their encoding nucleic acid sequences, are utilized in the construction of altered antibodies, preferably humanized antibodies, of this invention, by the following process. The same or similar techniques may also be employed to generate other embodiments of this invention.

A hybridoma producing a selected donor mAb, e.g., the mouse antibody 14F3, is conventionally cloned, and the DNA of its heavy and light chain variable regions obtained by techniques known to one of skill in the art, e.g., the techniques described in Sambrook et al., (Molecular Cloning (A Laboratory Manual), 2nd edition, Cold Spring Harbor Laboratory (1989)). The variable heavy and light regions of 14F3 containing at least the CDR-encoding regions and those portions of the acceptor mAb light and/or heavy variable domain framework regions required in order to retain donor mAb binding specificity, as well as the remaining immunoglobulin-derived parts of the antibody chain derived from a human immunoglobulin can be obtained using polynucleotide primers and reverse transcriptase. The CDR-encoding regions are identified using a known database and by comparison to other antibodies.

A mouse/human chimeric antibody may then be prepared and assayed for binding ability. Such a chimeric antibody contains the entire non-human donor antibody V_{H} and V_{L} regions, in association with human Ig constant regions for both chains.

A humanized antibody may be derived from the chimeric antibody, or preferably, made synthetically by inserting the donor mAb CDR-encoding regions from the heavy and light chains appropriately within the selected heavy and light chain framework. Alternatively, a humanized antibody of the invention may be prepared using standard mutagenesis techniques. Thus, the resulting humanized antibody contains human framework regions and donor mAb CDR-encoding regions. There may be subsequent manipulation of framework residues. The resulting humanized antibody can be expressed in recombinant host cells, e.g., COS, CHO or myeloma cells. Other humanized antibodies may be prepared using this technique on other suitable RANK-L-specific, neutralizing, high affinity, non-human antibodies.

A conventional expression vector or recombinant plasmid can be produced by placing these coding sequences for the altered antibody in operative association with conventional regulatory control sequences capable of controlling the replication and expression in, and/or secretion from, a host cell. Regulatory sequences include promoter sequences, e.g., CMV promoter, and signal sequences, which can be derived from other known antibodies. Similarly, a second expression vector can be produced having a DNA sequence which encodes a complementary antibody light or heavy chain. Preferably this second expression vector is identical to the first except insofar as the coding sequences and selectable markers are concerned, so to ensure as far as possible that each polypeptide chain is functionally expressed. Alternatively, the heavy and light chain coding sequences for the altered antibody may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The humanized antibody which includes the association of both the recombinant heavy chain and/or light chain is screened from culture by appropriate assay, such as ELISA or RIA. Similar conventional techniques may be employed to construct other altered antibodies and molecules of this invention.

Suitable vectors for the cloning and subcloning steps employed in the methods and construction of the compositions of this invention may be selected by one of skill in the art. For example, the conventional pUC series of cloning vectors, may be used. One vector used is pUC19, which is commercially available from supply houses, such as Amersham (Buckinghamshire, United Kingdom) or Pharmacia (Uppsala, Sweden). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

Similarly, the vectors employed for expression of the engineered antibodies according to this invention may be selected by one of skill in the art from any conventional vector. The vectors also contain selected regulatory sequences (such as CMV promoters) which direct the replication and expression of heterologous DNA sequences in selected host cells. These vectors contain the above described DNA sequences which code for the engineered antibody or altered immunoglobulin coding region. In addition, the vectors may incorporate the selected immunoglobulin sequences modified by the insertion of desirable restriction sites for ready manipulation.

The expression vectors may also be characterized by genes suitable for amplifying expression of the heterologous DNA sequences, e.g., the mammalian dihydrofolate reductase gene (DHFR). Other preferable vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the engineered antibodies or altered immunoglobulin molecules thereof. Host cells useful for the cloning and other manipulations of these cloning vectors are also conventional. However, most desirably, cells from various strains of E. coli are used for replication of the cloning vectors and other steps in the construction of altered antibodies of this invention.

Suitable host cells or cell lines for the expression of the engineered antibody or altered antibody of the invention are preferably mammalian cells such as CHO, COS, a fibroblast cell (e.g., 3T3), and myeloid cells, and more preferably a CHO or a myeloid cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs of the present invention (see, e.g., Pluckthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and references cited therein.

The general methods by which the vectors of the invention may be constructed, the transfection methods required to produce the host cells of the invention, and culture methods necessary to produce the altered antibody of the invention from such host cell are all conventional techniques. Likewise, once produced, the altered antibodies of the invention may be purified from the cell culture contents according to standard procedures of the art, including ammonium sulfate precipitation, affinity columns, column chromatography, gel electrophoresis and the like. Such techniques are within the skill of the art and do not limit this invention.

Yet another method of expression of the humanized antibodies may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

Once expressed by the desired method, the engineered antibody is then examined for in vitro activity by use of an appropriate assay. Presently conventional ELISA assay formats are employed to assess qualitative and quantitative binding of the engineered antibody to RANK-L. Additionally, other in vitro assays may also be used to verify neutralizing efficacy prior to subsequent human clinical studies performed to evaluate the persistence of the engineered antibody in the body despite the usual clearance mechanisms.

Following the general procedures described for preparing humanized antibodies, one of skill in the art may also construct humanized antibodies from other donor RANK-L antibodies, variable region sequences and CDR peptides described herein. Engineered antibodies can be produced with variable region frameworks potentially recognized as “self” by recipients of the engineered antibody. Minor modifications to the variable region frameworks can be implemented to effect large increases in antigen binding without appreciable increased immunogenicity for the recipient. Such engineered antibodies may effectively treat a human for RANK-L mediated conditions. Such antibodies may also be useful in the diagnosis of such conditions.

VII. Therapeutic/Prophylactic Uses

This invention also relates to a method of treating humans experiencing osteopenic diseases, including rheumatoid arthritis (RA), osteoporosis (OP), metastatic and primary bone cancer, wear debris induced osteolysis or osteoarthritis (OA), or immune diseases, including psoriasis, insulin dependent, diabetes (IDDM), inflammatory bowel disease (IBD), or multiple sclerosis (MS), which comprises administering an effective dose of antibodies including one or more of the engineered antibodies or altered antibodies described herein, or fragments thereof.

The therapeutic response induced by the use of the molecules of this invention is produced by the binding to human RANK-L and thus subsequently inhibiting osteoclast and dendritic cell development and function. Thus, the molecules of the present invention, when in preparations and formulations appropriate for therapeutic use, are highly desirable for those persons experiencing disorders of bone homeostasis, such as but not limited to osteopenic diseases, including rheumatoid arthritis (RA), osteoporosis (OP), metastatic and primary bone cancer, wear debris induced osteolysis or osteoarthritis (OA), and immune diseases, including psoriasis, insulin dependent, diabetes (IDDM), inflammatory bowel disease (IBD), or multiple sclerosis (MS). The molecules of the present invention, when in preparations and formulations appropriate for therapeutic use, are also highly desirable for those persons experiencing, including rheumatoid arthritis (RA), osteoporosis (OP), metastatic and primary bone cancer, wear debris induced osteolysis wear debris induced osteolysis or osteoarthritis (OA), or immune diseases, including psoriasis, insulin dependent, diabetes (IDDM), inflammatory bowel disease (IBD), or multiple sclerosis (MS).

The antibodies and fragments thereof of this invention may also be used in conjunction with cytokine inhibiting agents such as Cytokine Suppressive Anti-Inflammatory Drugs (CSAIDS™) or other antibodies, particularly human mAbs reactive with other markers (epitopes) responsible for the condition against which the antibody of the invention is directed.

The therapeutic agents of this invention are believed to be desirable for treatment of osteopenic or autoimmune conditions from about 2 days to 6 months or as needed. For example, longer treatments may be desirable when treating osteopenic diseases, including rheumatoid arthritis (RA), osteoporosis (OP), metastatic and primary bone cancer, wear debris induced osteolysis or osteoarthritis (OA), or immune diseases, including psoriasis, insulin dependent, diabetes (IDDM), inflammatory bowel disease (IBD), or multiple sclerosis (MS). The dose and duration of treatment relates to the relative duration of the molecules of the present invention in the human circulation, and can be adjusted by one of skill in the art depending upon the condition being treated and the general health of the patient.

The mode of administration of the therapeutic agent of the invention may be any suitable route which delivers the agent to the host. The antibodies and fragments thereof, and pharmaceutical compositions of the invention are particularly useful for parenteral administration, i.e., subcutaneously, intramuscularly, intravenously, or intranasally.

Therapeutic agents of the invention may be prepared as pharmaceutical compositions containing an effective amount of an antibody (e.g., humanized) of the invention as an active ingredient in a pharmaceutically acceptable carrier. In the prophylactic agent of the invention, an aqueous suspension or solution containing an antibody, preferably buffered at physiological pH, in a form ready for injection is preferred. The compositions for parenteral administration will commonly comprise a solution of the antibody of the invention or a cocktail thereof dissolved in an pharmaceutically acceptable carrier, preferably an aqueous carrier. A variety of aqueous carriers may be employed, e.g., 0.4% saline, 0.3% glycine, and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of an antibody of the invention. Similarly, a pharmaceutical composition of the invention for intravenous infusion could be made up to contain about 250 ml of sterile Ringer's solution, and about 1 to about 30 and preferably 5 mg to about 25 mg of an antibody of the invention. Actual methods for preparing parenterally administrable compositions are well known or will be apparent to those skilled in the art and are described in more detail in, for example, Remington's Pharmaceutical Science, 15th ed., Mack Publishing Company, Easton, Pa.

It is preferred that the therapeutic agent of the invention, when in a pharmaceutical preparation, be present in unit dose forms. The appropriate therapeutically effective dose can be determined readily by those of skill in the art. To effectively treat an inflammatory disorder in a human or other animal, one dose of approximately 0.1 mg to approximately 20 mg per 70 kg body weight of a protein or an antibody of this invention should be administered parenterally, preferably i.v. or i.m. (intramuscularly). Such dose may, if necessary, be repeated at appropriate time intervals selected as appropriate by a physician during the disease.

The antibodies of this invention may also be used in diagnostic regimens, such as for the determination of RANK-L mediated disorders or tracking progress of treatment of such disorders. As diagnostic reagents, these antibodies may be conventionally labeled for use in ELISA's and other conventional assay formats for the measurement of RANK-L levels in serum, plasma or other appropriate tissue, or the release by human cells in culture. The nature of the assay in which the antibodies are used are conventional and do not limit this disclosure.

Thus, one embodiment of the present invention relates to a method for aiding the diagnosis of disorders of bone homeostasis or autoimmune disease and other conditions associated with excess or deficient osteoclast or T cell activity (e.g. osteopenic diseases, including rheumatoid arthritis (RA), osteoporosis (OP), metastatic and primary bone cancer, wear debris induced osteolysis or osteoarthritis (OA), and immune diseases, including psoriasis, insulin dependent, diabetes (IDDM), inflammatory bowel disease (IBD), or multiple sclerosis (MS), etc.) in a patient which comprises the steps of determining the amount of human RANK-L in sample (plasma or tissue) obtained from said patient and comparing said determined amount to the mean amount of human RANK-L in the normal population, whereby the presence of a significantly elevated amount of RANK-L in the patient's sample is an indication of bone or autoimmune disease and other conditions associated with excess osteoclast or T cell number or activity. Similarly, the presence of a significantly reduced amount of RANKL L in the patient's sample is an indication bone disease associated with deficient osteoclast number or activity.

The antibodies or fragments thereof described herein can be lyophilized for storage and reconstituted in a suitable carrier prior to use. This technique has been shown to be effective with conventional immunoglobulins and art-known lyophilization and reconstitution techniques can be employed.

Thus, the present application relates to (a) a monoclonal antibody that binds to human RANK-L; (b) a monoclonal antibody has the identifying characteristics of monoclonal antibody 14F3; and (c) the monoclonal antibody 14F3.

The present invention also relates to (a) an isolated polypeptide comprising an immunoglobulin complementarity determining region of the antibody that binds to human RANK-L; (b) isolated polypeptide comprising an immunoglobulin complementarity determining region of the antibody characteristics of monoclonal antibody 14F3; (c) an isolated polypeptide comprising an immunoglobulin complementarity determining region of monoclonal antibody of 14F3. The present invention also relates to an isolated polynucleotide comprising the polynucleotide encoding the polypeptide of (a), (b), and (c).

The polypeptide of the present invention relates, among others, to the immunoglobulin complementarity determining region that comprises the polypeptide selected from the group consisting of SEQ ID NOs:5, 6, 7, 8, 9 and 10. The polynucleotide of the invention relates to, among others, polynucleotide comprising the polynucleotide encoding polypeptide selected from the group consisting of SEQ ID NOs:5, 6, 7, 8, 9 and 10. The present application relates to (a) a monoclonal antibody that binds to human RANK-L wherein the immunoglobulin complementarity determining region comprises the polypeptides selected from the group consisting of SEQ ID NOs: 5, 6, 7, 8, 9, and 10; (b) a monoclonal antibody comprising a heavy chain variable region polypeptide as set forth in SEQ ID NO: 2, and/or light chain variable region polypeptide as set forth in SEQ ID NO: 4.

The polynucleotide of the present invention also relates to an isolated polynucleotide comprising a polynucleotide encoding a polypeptide selected from the group consisting of SEQ ID NO:2 and SEQ ID NO:4.

The present invention relates to a hybridoma cell line that produces a monoclonal antibody having the identifying characteristics the monoclonal antibody 14F3. Also included is a pharmaceutical composition comprising (a) a monoclonal antibody that binds to human RANK-L; (b) a monoclonal antibody has the identifying characteristics of monoclonal antibody 14F3; and (c) the monoclonal antibody 14F3.

The present invention relates to a method for detecting the presence human RANK-L in a sample which comprises:

a) exposing the sample to an antibody that binds to human RANK-L; and

b) detecting the antibody that is bound to human RANK-L.

Among the preferred method is wherein the sample is treated before exposure to the antibody such that the human RANK-L protein is accessible to binding by the antibody. The preferred antibody that binds to human RANK-L has the identifying characteristics of monoclonal antibody 14F3, which even more preferably is monoclonal antibody 14F3.

The following examples illustrate various aspects of this invention, and are not to be construed as limiting the scope of this invention. All amino acids are identified by conventional three letter or single letter codes. All necessary restriction enzymes, plasmids, and other reagents and materials were obtained from commercial sources unless otherwise indicated. All general cloning legation and other recombinant DNA methodology were as performed in T. Maniatis et al., cited above, or the second edition thereof (1989), eds. Sambrook et al., by the same publisher (“Sambrook et al.”).

### BIOLOGICAL METHODS/EXAMPLES

### Example 1

### Production of MAbs to RANK-L

A. Monoclonal antibody generation

The monoclonal antibodies were generated by immunizing CB6 fl mice with multiple does of soluble human RANKL protein. Antisera were taken from the immunized mice and titered for anti-RANKL antibody. On the basis of the test bleed immunoassay, the best responding mouse was boosted at 3 and 1 days prior to spleenectomy. The spleen was removed and the spleen cells fused with X63 AG8 653 myeloma cells using polyethylene glycol methodology. The fused cells were then cultured in 20×96 well tissue culture plates. After 14 days post fusion the hybridomas were assayed for antibody binding to RANKL protein. Those hybridomas with antibody binding to RANKL were expanded to progressively larger tissue culture plates according to the growth rate of the hybridoma. Supernatant from the hybridomas was used in immunoassays to confirm the antibody specificity and its biological activity in neutralizing RANKL/RANK binding. Once confirmed the hybridoma cell line was cryopreserved and scaled for antibody production in serum free media.

A great problem in the generation of antibodies to the RANKL protein was apoptosis of the hybridoma cultures. This normally occurred in the early stages of hybridoma expansion and resulted in either the death of the cell line completely or the generation of non-producer hybridoma cell lines that had switched off antibody synthesis. This problem was rather unique to the RANKL antigen relative to our observations with over 100 other antigens. This effect perhaps results from weak cross reactivity of the RANKL antibodies to murine RANKL. If RANKL is present on the hybridoma cells, the relatively high concentration of the RANKL antibody in the hybridoma culture medium may lead to binding to RANKL induction of apoptosis. The addition of hybridoma growth factors to stimulate growth and offset the apoptosis effects was tried but proved ineffective with most of the hybridoma cell lines. The outcome of this problem was either cell line death or IgG synthesis shutdown with greater than 90% of the hybridomas being lost from the fusion. In some fusions all the hybridomas were lost in this manner.

To combat this effect, multiple mice were immunized and the spleens successively used in order to generate a panel of stable hybridomas secreting anti-RANKL antibodies for evaluation in biological assays.

B. Purifcation and Sequencing of the 14F3 Mab

The 14F3 Mab was purified by ProsepA (Bio Processing, Consett, UK) chromatography respectively using the manufacturer's instructions. The Mab was >95% pure by SDS-PAGE. For N-terminal sequence analysis, the heavy and light chain polypeptides were separated by SDS-PAGE, transferred to a PVDF (polyvinylidene difluoride) membrane and sequenced directly (P. Matsudaira J. Biol. Chem. 262: 10035-10038, 1987).

C. Isotyping of Mabs

The murine RANKL mAb 14F3 was isotyped by commercially available kits (Zymed, Amersham) and found to be IgG2a/kappa.

### Example 2

### Assays

A. A competition ELISA was established using a human RANK-Fc fusion protein coated on plastic and a biotinylated soluble human RANKL protein for detection in solution. The RANK-FC and RANKL proteins were produced in CHO cells and Pichia pastoris, respectively, and purified to >90% homogeneity. The shRANKL (soluble, human RANKL) protein was biotinylated at a 20:1 molar ratio of NHS-biotin to protein (Pierce, Rockford, Ill.) according to the manufacturer's specifications. 96-well ELISA plates were coated overnight at 4° C. with 50 ng/well (0.53 nmols) RANK-Fc in pH 9.6 carbonate-bicarbonate buffer. Plates were washed in pH 7.4 Tris-Saline buffer containing 0.1% Tween 20 and blocked for 2 h at RT in 1% BSA/PBS. Competitor proteins (RANK-Fc; death receptor 5 (DR5)-Fc; OPG-Fc; RANKL mAb 14F3) were diluted in 0.01% Tween 20/PBS and added to wells prior to the addition of biotinylated shRANKL (0.43 nM) and the combined samples were incubated for 2 hrs at room temperature. The amount of biotinylated shRANKL bound to coated RANK-Fc (+/−competitor) was measured using alkaline phosphatase conjugated streptavidin. The substrate for signal detection was 105 PNPP (Pierce Inc., Rockford, Ill.) and absorbence measured at 405 nm using a Spectra Max 340 plate reader. The DR5-Fc protein showed no inhibition, as expected from various other studies indicating that it did not interact with RANKL. In several different parallel assays, OPG-Fc was a more potent inhibitor than RANKL-Fc, with IC50's of about 0.5 and 6 nM, respectively. The 14F3 mAb showed a potency more similar to that of OPG-Fc with an IC50 of about 2 nM.

B. Inhibition of maturation of human monocyte-derived dendritic cells in culture. Fresh human monocytes purified by gradient isolation were treated for 6 days in culture with recombinant human 1L-4 (25 ng/ml) and human GM-CSF (SOng/ml) to generate dendritic cells with the antigen capturing phenotype (immature DC). The media was changed on day 6 with the addition of either recombinant human TNFa (30 ng/ml) or soluble RANKL (30 ng/ml) in the presence or absence of the TNFα antagonist TNFR11-Fc (30 ug/ml) or the RANKL mAb 14F3 (30 ug/ml). TNFα or RANKL alone induced the formation of mature DC as measured by phenotypic, morphological, and functional properties. Thus, the cells showed upregulation of cell surface CD83, CD86, CD80, and MHC II and down modulation of CD1a. Whereas immature cells showed marked uptake of FITC-dextran, which is indicative of macropinocytosis, mature cells had virtually lost this capability. TNFα was more effective than RANKL in inducing maturation, resulting in essentially a homogeneous population of mature DCs. In contrast, treatment with RANKL produced population of cells of similar phenotype, but only a fraction of the cells (30-80% in different experiments with monocytes obtained from different donors) showed this phenotype in cells treated with RANKL. The RANKL mAb 14F3 blocked the maturation of cells treated with sRANKL but had no effect on cells treated with TNFα. Similarly, TNFR1-Fc blocked the maturation of cells treated with TNFa but had no effect on cells treated with sRANKL. Thus, RANKL mAb 14F3 specifically inhibits the functional activity of RANKL induction of DC maturation.

C. Inhibition of human sRANKL-stimulated bone marrow murine osteoclastogenesis in cell culture. Bone marrow cells were collected from the femurs of 6 week old Balb/C mice, washed 3 times, counted, then resuspended in medium (RPMI plus 10% FCS, glutamine, penicillin/streptomycin and 25 ng/ml CSF-1, 50 ng/ml soluble RANKL). These cells were plated at 5×10⁵/well in Nunc 24-well multiwell plates (in quadruplicate) and cultured (37° C., 5% CO₂) for 7-10 days. Test agents (e.g. antibodies, OPG-Fc) were added at the initiation of the culture. Medium and test agents were replaced every 3-4 days. At the end of the culture period, the cells were fixed and stained for tartrate-resistant acid phosphatase (TRAP) using Sigma kit 386-1 in accordance with the manufacturer's instructions. The number of osteoclasts (defined as TRAP positive cells with ≧3 nuclei) in each well were enumerated microscopically. Both the RANKL mAb 14F3 and OPG-Fc completely inhibited osteoclastogenesis, as measured by the number of osteoclasts developing per well, at a concentration of 1 ug/ml and both showed an IC50 in this assay of about 200 ng/ml. In contrast, a RANK-Fc fusion protein fully inhibited at 10 ug/ml but was without effect at 2 ug/ml.

D. Inhibition of Human Monocyte Osteoclastogenesis Mediated by RANKL.

Human monocytes were prepared as described for dendritic cell maturation in section B above. Culturing of these cells for 6-8 days in the presence of 50 ng/ml human soluble RANKL plus 25 ng/ml of human M-CSF led to the formation of osteoclasts with bone resorbing activity. For inhibition studies, the RANKL mAb 14F3 or RANK-Fc protein was added at the initiation of culture and the formation of osteoclasts was monitored by formation of multinuclear cells. In one assay, the 14F3 mAb gave an IC50 of about 4 ug/ml whereas the RANK-Fc protein was more active with an IC50 of about 500 ng/ml, in contrast to the observations in the murine osteoclastogenesis assay (part C above).

In summary, these results show that RANKL mAb 14F3 is a potent inhibitor of the interaction between human RANKL and its receptor RANK. This inhibition leads to antagonism of RANKL-mediated DC maturation and osteoclast development and function.

### Example 3

### CDR sequences

Gene Cloning and Sequence Analysis:

The variable heavy and light genes were cloned from hybridoma cells using standard molecular biological methods described briefly as follows. Total RNA was isolated from the hybridoma cells using TRIzol Reagent (Life Technologies Cat. # 15596-026) according to manufacturer's protocol. The RNA was reverse transcribed with a RT-PCR kit per the manufacturer's instructions (Boehringer Mannheim Cat. No. 1483-188) using a poly-dT oligonucleotide for priming. Following first strand cDNA synthesis, the heavy and light V regions were PCR amplified using 3′ constant region specific primers and degenerate 5′ primers. The degenerate 5′ primer sequences were designed to encode the previously determined N terminal amino acid sequences of the variable heavy or light chain regions. Full length sequences from multiple clones were obtained from each PCR amplification and aligned to provide consensus. Accordingly, the first 17 bases of DNA sequence for both the heavy and light chains are PCR primer generated, however the translated protein sequence is native.

What is claimed is:

1. Monoclonal antibody of that has the identifying characteristics of monoclonal antibody 14F3.

2. The monoclonal antibody of claim 1 that is monoclonal antibody 14F3.

3. An antibody comprising an immunoglobulin complementarity determining region of monoclonal antibody 14F3.

4. An isolated polynucleotide comprising the polynucleotide encoding the antibody of claim 3.

5. An antibody which comprises the polypeptides having the amino acid sequences of SEQ ID NOs:5, 6, 7, 8, 9 and 10.

6. An antibody which comprises the polypeptides having the amino acid sequences of SEQ ID NOs: 2 and 4.

7. Isolated polynucleotides which encode an antibody comprising the polypeptides having the amino acid sequences of SEQ ID NOs:5, 6, 7, 8, 9 and 10.

8. Isolated polynucleotides which encode an antibody comprising the polypeptides having the amino acid sequences of SEQ ID NOs: 2 and 4.

9. Expression systems comprising polynucleotides which encode an antibody comprising the polypeptides having the amino acid sequences of SEQ ID NOs:5, 6, 7, 8, 9 and 10 capable of producing such antibody when said expression vectors are present in a compatible host cell, and recombinant host cells comprising such expression vectors.

10. A process for producing an antibody which comprises the polypeptides having the amino acid sequences of SEQ ID NOs:5, 6, 7, 8, 9 and 10 comprising the step of culturing said host cells under conditions sufficient for the production of said antibody and recovering the antibody from the culture medium.

11. Expression systems comprising polynucleotides which encode an antibody comprising the polypeptides having the amino acid sequences of SEQ ID NOs: 2 and 4 capable of producing such antibody when said expression vectors are present in a compatible host cell, and recombinant host cells comprising such expression vectors.

12. A process for producing an antibody which comprises the polypeptides having the amino acid sequences of SEQ ID NOs:2 and 4 comprising the step of culturing said host cells under conditions sufficient for the production of said antibody and recovering the antibody from the culture medium.

13. A method of treating or preventing osteopenic diseases, including rheumatoid arthritis (RA), osteoporosis (OP), metastatic and primary bone cancer, wear debris induced osteolysis or osteoarthritis (OA), and immune diseases, including psoriasis, insulin dependent, diabetes (IDDM), inflammatory bowel disease (IBD), or multiple sclerosis (MS) by administering an effective dose of the antibody or polypeptide of claim 1, 2, 3, 5 or 5 to a patient in need thereof.
